(19) United States
(12) Patent Application Publication
(10) Pub. No.: US 2015/0099270 A1
(43) Pub. Date: Apr. 9, 2015

(54) METHOD OF SCREENING PHARMACEUTICALS FOR DRUG INTERACTIONS AND NEPHROTOXICITY

(71) Applicant: Thomas C. DOWLING, Forest Hill, MD (US)

(72) Inventor: Thomas C. DOWLING, Forest Hill, MD (US)

(21) Appl. No.: 14/507,391

(22) Filed: Oct. 6, 2014

Related U.S. Application Data
(60) Provisional application No. 61/886,762, filed on Oct. 4, 2013.

(51) Int. Cl. G01N 33/50

(52) U.S. Cl. CPC G01N 33/5014; G01N 33/5044; G01N 33/5038; G01N 33/5088; G01N 2570/00; G01N 2500/10; G01N 2333/91051
USPC 435/7.4; 435/34; 435/15

(57) ABSTRACT

A method of determining nephrotoxicity of pharmaceuticals by conducting a metabolite formation study in cells using PAH in a control group; measuring metabolite formation; exposing cells to pharmaceuticals in the treatment group; conducting the metabolite formation study using PAH; measuring the metabolite formation in the treatment group; comparing the metabolite formation in the control and treatment group, and making a determination as to the nephrotoxicity of pharmaceutical.

### CROSS-REFERENCE TO RELATED APPLICATION

This application claims the benefit of U.S. Provisional Application No. 61/886,762 filed Oct. 4, 2013.

### BACKGROUND OF THE INVENTION

Numerous pharmaceuticals and other substances are known to be nephrotoxic and can cause renal failure through a variety of mechanisms including direct toxicity to the renal tubules, allergic interstitial nephritis, and crystallization of the drug within the renal tubules, all of which can lead to acute oliguric renal failure. Nephrotoxicity can result from the administration of any pharmacological class as well as from the administration of one or more pharmacologic class to the same patient. The identification of cellular stress mechanisms is fundamental to understanding the susceptibility of the kidney to chemicals and pharmaceuticals and for the development of renal biomarkers indicative of sublethal injury.

Drug interactions between two or more drugs in the kidney can result in nephrotoxicity and alterations in the systemic drug concentrations of one or more interacting drugs. This could lead to reduced elimination of one or more drugs by the kidney, leading to excessive accumulation of a drug in the body and side effects. Thus it is important to screen for potential drugs that may cause changes in the function of the kidney in order to avoid serious medical problems.

### SUMMARY OF THE INVENTION

An important reaction in the metabolism of pharmaceuticals is arylamine N-acetyltransferse (NAT) catalyzing the reaction of acetyl-CoA with an arylamine that results in CoA and an N-acetylarylamine. This phase II reduction reaction is important because it is needed in the acetylation (and inactivation) of pharmaceuticals.

Para-aminohippuric acid (PAH) is a diagnostic agent used to measure renal plasma blood flow. Uptake of PAH into renal proximal tubule cells occurs via the Organic Anionic Transporter (OAT), with efflux at the apical membrane likely facilitated by the Multi-Drug Resistance Protein 2 (MRP2).

We have identified the formation of the N-acetylarylamine metabolite of PAH (n-acetylPAH, aPAH) in HK-2 cells. This chemical name of this metabolite is 2-[(4-acetamidobenzoyl)amino]acetic acid. The present invention involves quantifying NAT-mediated metabolism of PAH to determine the nephrotoxicity of one or more pharmaceuticals. This analysis is performed through in-vitro screening and/or through screening the in-vivo metabolism of PAH.

### BRIEF DESCRIPTION OF THE FIGURES

FIG. 1 is a microscopic image comprising cellular densities for (A) Day 5 post-seeding (100×), and (B) Day 5 post-seeding (40×) of HK-2 cell culture.

FIG. 2 are chemical structures of PAH and acetyl-PAH.

FIG. 3 is a representative plot of the amount of acetyl-PAH generation vs. time in HK-2 cells incubated with 200 μg/ml of PAH.

FIG. 4 is a representative plot of the urinary excretion of acetyl-PAH over time in a clinical study.

FIG. 5 is a representative plot of the plasma concentrations of acetyl-PAH over time in a clinical study.

FIG. 6 is a Western Blot image showing the detection of NAT1 enzyme using electrophoresis gel from HK-2 cell lysate

### DETAILED DESCRIPTION OF THE INVENTION

Drug metabolism is the irreversible chemical alteration of a drug by the body. The substances that result from metabolism (metabolites) may be inactive, or they may be similar to or different from the original drug in therapeutic activity or toxicity. Some drugs, called prodrugs, are administered in an inactive form, which is metabolized into an active form. The resulting active metabolites produce the desired therapeutic effects. Metabolites may be metabolized further or excreted quickly from the body. The subsequent metabolites are then excreted. Most drugs must pass through the liver, which is the primary site for drug metabolism. Once in the liver, enzymes convert prodrugs to active metabolites or convert active drugs to inactive forms. The liver's primary mechanism for metabolizing drugs is via Phase I or Phase II enzymes. Phase I reactions typically involve Cytochrome P450 enzymes (CYP), which is often followed by conjugation to polar compounds in phase II reactions. These reactions are catalyzed by transferase enzymes. In phase II reactions, these activated xenobiotic metabolites are conjugated with charged species such as glutathione, sulfate, glycine, or glucuronic acid. Sites on drugs where conjugation reactions occur include carboxyl (—COOH), hydroxyl (—OH), amino (NH₂), and sulfhydryl (—SH) groups. Products of conjugation reactions have increased molecular weight and tend to be less active than their substrates, unlike Phase I reactions which often produce active metabolites. The addition of large anionic groups (such as glutathione) detoxifies reactive electrophiles and produces more polar metabolites that cannot diffuse across membranes, and may, therefore, be actively transported.

The capacity of enzymes to metabolize is limited, so they can become overloaded when blood levels of a drug are high. Many substances (such as drugs and foods) affect drug metabolizing enzymes, leading to drug interactions. If these substances decrease the ability of the enzymes to break down a drug, then that drug's effects (including side effects) are increased. If the substances increase the ability of the enzymes to break down a drug, then that drug's effects are decreased. Drug interactions occur when two or more drugs compete with each other for a limited amount of enzyme or transporter, leading to a change in the pharmacokinetics or systemic exposure of one or more drugs. This type of drug-drug interaction may lead to an unexpected side effect.

An important reaction in the metabolism of pharmaceuticals is arylamine N-acetyltransferse (NAT). N-acetyltransferase is a cytosolic enzyme that catalyzes the transfer of acetyl groups from acetyl-CoA to arylamines. This enzyme catalyzes the Phase II conjugation reaction of acetyl-CoA with an arylamine that results in formation of Co-enzyme A (CoA) and an N-acetylarylamine.

The NAT-mediated phase II reduction reaction is important because it is needed in the acetylation (and inactivation) of pharmaceuticals containing aromatic and heterocyclic amines. NAT enzymes can either facilitate the detoxification of carcinogenic arylamines into innocuous metabolites by N-acetylation or promote their metabolic activation into DNA-binding electrophiles via O-acetylation.

Para-aminohippuric acid (PAH) is a diagnostic agent used to measure effective renal plasma blood flow (ERPF). The underlying principle for using PAH to measure RPF is that PAH is extracted by the kidney tubule cells, such that its elimination into urine is directly proportional to the blood flow supply to the kidney. PAH is an organic anion that must be actively taken up into kidney cells. The interior of a renal proximal tubule cell is negatively charged (−70 mV), providing a natural barrier or repelling force to keep anionic substances (such as PAH) from getting inside the cell. Thus, in order for an anionic substance (or drug) to get into the cell, an active uptake mechanism at the cell surface (or transporter) is required. For example, uptake of the anionic compound PAH into renal proximal tubule cells occurs via the OAT transporter. This uptake is highly efficient, such that PAH is nearly completely extracted by the kidney in a single pass of blood flow through the kidney. Thus, PAH is often used as a diagnostic marker for kidney blood flow, where renal excretion of PAH is directly correlated with the rate of blood flow through the kidney. The cumulative renal extraction of PAH is likely a combination of OAT-mediated uptake (at basolateral membrane) and efflux out of the cell at the apical membrane (into the urine) by other transporters such as Multi-Drug Resistance Protein 2 (MRP2). The location of the MRP2 transporter leads active transport of PAH from the interior of the kidney cell into the lumen or urine flow. Thus, net tubular secretion of organic anionic drugs such as PAH occurs by active, energy-dependent transport process.

PAH has been used as a diagnostic marker to evaluate disease- and drug-related effects on renal tubular secretion. Data from renal cell culture experiments suggest that intracellular mediators such as protein kinase C, which is elevated in chronic renal disease, may regulate anionic secretion. In experimental models of chronic and acute renal failure, differential effects on the renal extraction of PAH (anion) and tetraethylammonium (TEA, cation) have been reported. For example, in dogs with azotemia induced by bilateral ureteral-venous anastomosis, the extraction of PAH was significantly lower than that for TEA (0.57 vs. 0.80, p=0.025) suggesting that the anionic pathway may be preferentially affected by renal disease due to either selective injury or through competition with circulating endogenous anions.

PAH is nearly completely extracted by the human kidney and is commonly used in clinical research investigations to measure effective renal plasma flow (ERPF).

Other agents such as phenol red, ortho-iodohippurate (Hippuran), and phenolsulfonthalein (PSP) which are also extensively secreted by the anionic pathway have been proposed but are less commonly used to measure ERPF. The renal clearance of PSP underestimates ERPF and thus it is not a valid quantitative marker of ERPF. However, it has been observed that NAT-mediated metabolites such as acetyl-PAH appear in high quantities in the urine after PAH administration. Thus, based on the high intrinsic clearance of PAH, due in part to secretion via the OAT anionic pathway we investigated the appearance of plasma and urinary acetyl-PAH to understand the role of renal tubular secretion (See Example 6).

HK-2 cells are derived from human proximal tubule cells that appear to maintain many characteristics of an intact human kidney. The HK-2 cell line is an immortalized line derived from the human epithelial renal proximal tubule. The original cells were isolated from the cortical proximal tubule segment, cultured and exposed to a recombinant virus containing the E6 and E7 genes of HPV16. A cell clone, in which PCR analysis confirmed the incorporation of HPV16 E6/E7 construct in genomic DNA, was designed as HK-2 and was able to continuously grow for more than a year. The HK-2 cells have many of the same characteristics of adult normal tubular cells, including brush border enzyme activity (acid and alkaline phosphatase, leucine aminopeptidase, gammaglutamyltranspeptidase) and the efflux protein P-glycoprotein (PGP). Although little is known regarding intra-cellular enzyme activity in HK-2 cells, such knowledge would be useful for studying intra-cellular drug metabolism, intra-cellular drug toxicity and nephrotoxicity.

We have identified the formation of the N-acetylarylamine metabolite of PAH (n-acetyl-PAH) in HK-2 cells. This chemical name of this metabolite is 2-[(4-acetamidobenzoyl)amino]acetic acid. This metabolite is a product of the NAT enzyme, and its presence in the HK-2 cell system indicates that it can be used as a marker of NAT activity in kidney cells. Measurement of NAT activity in the kidney can be used to further study the mechanisms of drug-drug interactions in the kidney, toxicity of drugs in the kidney, and handling of pro-carcinogenic agents in the kidney.

The present invention involves the discovery that quantifying NAT-mediated metabolism of PAH corresponds to nephrotoxicity resulting from the exposure to one or more pharmaceuticals.

The term “nephrotoxicity” is meant for purposes of the present invention to mean a deleterious effect of some substances, both toxic chemicals and medication, on the kidneys or kidney tissues. Drugs are a common source of injury to the kidneys (called nephrotoxicity), including community and hospital-acquired acute kidney failure. Among older adults, the incidence of drug-induced nephrotoxicity may be very high, likely related to the high incidence of diabetes and cardiovascular disease, use of multiple co-medications, and exposure to diagnostic and therapeutic procedures with the potential to harm the kidneys. Although renal impairment is often reversible if the offending drug is discontinued, the condition can be costly and may require multiple interventions, including hospitalization. Most drugs that are associated with causing nephrotoxicity exert toxic effects by specific cellular mechanisms. These include altered blood flow in the kidney (vasoconstriction), tubular cell toxicity, inflammation, crystal formation, rhabdomyolysis, and thrombotic microangiopathy. Knowledge of offending drugs and the mechanism by which they cause injury to the kidney is critical to recognizing and preventing drug-induced renal impairment.

This analysis is performed through in-vitro screening and/or through screening the in-vivo metabolism of PAH. Nephrotoxicity may comprise a statistically significant (p<0.05) change in metabolism of a probe substrate in the presence of the offending drug, when compared to a non-treated control group.

Determination of In Vitro Drug-Drug Interactions take into account findings from in vitro metabolism, transport, and drug interaction studies and are valuable in quantitatively assessing the drug-drug interaction potential of an investigational drug. Along with clinical pharmacokinetic data, results from in vitro studies can serve as a screening mechanism to rule out the need for additional in vivo studies, or provide a mechanistic basis for proper design of clinical studies using a modeling and simulation approach. In vitro drug-drug interaction studies must be performed with high quality and consistency, particularly when the studies ultimately influence the design of clinical trials. The experimental procedures and documentation of data for in vitro work should be rigorous, reproducible, with specific analytical methods and documentation of assay procedures and results.

To determine whether a drug-drug interaction is present, such as inhibition of a particular enzyme (i.e. NAT), changes in the metabolism of a NAT-specific substrate (probe substrate such as PAH) by human kidney cells expressing NAT with varying concentrations of the interacting drug are monitored. The observation of a statistically significant change in metabolite formation (p<005) of the probe substrate indicates the presence of a metabolic drug-drug interaction. Potency of the inhibition and rank order of the inhibition of different enzymes can be assessed by the determination of the Kᵢ or IC₅₀ value (drug concentration, which reduced the metabolism of the NAT probe substrate by 50%). The concentration of probe substrate used should be at or below its Michaelis-Menten constant (Kₘ). Therefore, before performing in vitro inhibition studies with new drugs, the test system (e.g., HK-2 cells) needs to be established, and kinetic parameters of the NAT probe substrate (Kₘ, Vₘₐₓ), as well as inhibition (Kᵢ or IC₅₀) by a typical NAT inhibitor determined and compared with reference values.

Determination of In Vivo (Human) Interactions takes into account the fact that two drugs sharing a common metabolic or transporter pathway does not guarantee that they will have a clinically significant pharmacokinetic interaction when co-administered to a patient. Whether the two co-administered drugs will interact in humans will depend on various factors, including the relative affinities of each drug for the binding site on the metabolizing enzyme or transporter as well as the effective free drug concentrations available locally for binding. In addition, parallel pathways for elimination of one or both drugs would tend to reduce the potential for a significant pharmacokinetic interaction. The magnitude of the elevation in systemic exposure (i.e. blood concentrations) or reduced urinary excretion of a drug or metabolite as a result of inhibition or alterations of the enzymes or transporters responsible for its metabolism by a co-administered inhibitor (pharmaceutical precipitant) will depend on the degree of inhibition of the relevant metabolic enzyme or transporter by the precipitant. The presence of a drug-drug interaction comprises a statistically significant (p<0.05) change in a pharmacokinetic parameter of a probe substrate or a metabolite (i.e. AUC or CL), with >30% change from baseline most likely to infer clinical significance. The presence of such a drug-drug interaction may not necessarily lead to a clinical consequence. For example, when a drug is co-administered with a known inhibitor of an enzyme responsible for its transport or metabolism, the resulting increased systemic exposure to the recipient drug, and its metabolites may not necessarily result in a clinically detectable increase in toxicity. Whether a given magnitude of effect of an interacting inhibitory pharmaceutical on plasma levels of a recipient pharmaceutical results in an increased risk of adverse events depends to a great extent on the therapeutic index (i.e., ratio of efficacy to toxicity) of the recipient drug. Even small pharmacokinetic interactions can result in significant pharmacodynamic adverse effects for pharmaceuticals that have typical therapeutic concentrations close to those associated with toxicity. However, small to moderate pharmacokinetic interactions may not necessarily result in detectable and clinically significant consequences for drugs that have a wider therapeutic index. Clinical data regarding the safety of co-administration of a drug with another potentially interacting drug, when available from clinical trials and from post-marketing surveillance, are always more relevant and definitive in assessing the clinical relevance of a proven or potential pharmacokinetic drug interaction than the pharmacokinetic data in itself, i.e., when sufficient clinical experience exists, clinical data always takes precedence over pharmacokinetic data in terms of establishing the clinical significance of drug-drug interactions.

The nephrotoxicity of pharmaceuticals from multiple pharmacologic classes may be screened using the proposed invention. The presence of nephrotoxicity may comprise a statistically significant (p<0.05) change in metabolism of a probe substrate in the presence of the offending pharmaceutical, when compared to a non-treated control group. These pharmacologic classes include and are not limited to pharmaceuticals identified as antihistamines, anti-infectives, antineoplastics, autonomic agents, blood derivatives, blood coagulation thrombotic agents, cardiovascular agents, CNS agents, contraceptives, dental agents, dietary supplements, electrolytes, enzymes, respiratory agents, EENT preparations, gastrointestinal agents, gold and heavy metal compounds hormones (and synthetic hormones), anesthetics oxytocics, radioactive agents, vaccines, and immunologics.

The term “pharmaceutical” or “drug” is meant for purposes of the present invention to mean a pharmaceutical drug or medicinal product is any chemical substance formulated or compounded as a single active ingredient product intended for internal, or external or for use in the medical diagnosis, cure, treatment, or prevention of disease. These pharmaceuticals include drugs in the classes listed above as well as the following: Abacavir Sulfate, Abatacept, Acamprosate Calcium, Acarbose, Acebutolol Hydrochloride, Acetaminophen, Acetohydroxamic Acid, Acetylcysteine, Acrivastine, Acyclovir, Acyclovir Sodium, Adalimumab, Adefovir Dipivoxil, Adenosine Phosphate, Agalsidase Beta, Albendazole, Alendronate Sodium, Alglucosidase Alfa, Aliskiren Hemifumarate, Allopurinol, Almotriptan Malate, Alogliptin Benzoate, Alosetron Hydrochloride, Alprazolam, Alprostadil, Alvimopan, Amantadine Hydrochloride, Ambenonium Chloride, Ambrisentan, Amifostine, Amikacin Sulfate, Amiloride Hydrochloride, Aminohippurate Sodium, Aminosalicylic Acid, Amitriptyline Hydrochloride, Amlodipine Besylate, Ammonia Spirit, Aromatic, Ammonium Chloride, Amobarbital, Amobarbital Sodium, Amoxapine, Amoxicillin, Amoxicillin and Clavulanate Potassium, Amphetamine Aspartate, Amphetamine Sulfate, Amphotericin B, Ampicillin Sodium and Sulbactam Sodium, Ampicillin, Ampicillin Sodium, Ampicillin Trihydrate, Amyl Nitrite, Anakinra, Anidulafungin, Anthraquinone Laxatives, Antithymocyte Globulin, Aprepitant/Fosaprepitant Dimeglumine, Arginine Hydrochloride, Aripiprazole, Armodafinil, Asenapine Maleate, Aspirin, Atazanavir Sulfate, Atenolol, Atomoxetine Hydrochloride, Atorvastatin Calcium, Auranofin, Aurothioglucose, Gold Sodium Thiomalate, Azathioprine, Azathioprine Sodium, Azilsartan Kamedoxomil, Azithromycin, Aztreonam, Bacitracin, Balsalazide Disodium, Basiliximab, Beclomethasone Dipropionate, Bedaquiline Fumarate, Belatacept, Belimumab, Benazepril Hydrochloride, Bendroflumethiazide, Benzonatate, Benzphetamine Hydrochloride, Beractant, Betaine, Betamethasone, Betamethasone Acetate, Betamethasone Sodium Phosphate, Betaxolol Hydrochloride, Bethanechol Chloride, Bisacodyl, Bismuth Salts, Bisoprolol Fumarate, Bosentan, Botulinum Toxin, Brompheniramine Maleate, Dexbrompheniramine Maleate, Budesonide, Bulk-Forming Laxatives, Bumetanide, Buprenorphine, Buprenorphine Hydrochloride, Bupropion Hydrochloride, Buspirone Hydrochloride, Butabarbital Sodium, Butorphanol Tartrate, Caffeine, Caffeine and Sodium Benzoate Injection, Caffeine, Citrated, Calcitonin, Canagliflozin, Canakinumab, Candesartan Cilexetil, Capreomycin Sulfate, Captopril, Carbamazepine, Carbinoxamine Maleate, Carboprost Tromethamine, Carglumic Acid, Carvedilol, Caspofungin Acetate, Castor Oil, Cautions, Cefaclor, Cefadroxil, Cefazolin Sodium, Cefdinir, Cefditoren Pivoxil, Cefepime Hydrochloride, Cefixime, Cefotaxime Sodium, Cefotetan Disodium, Cefoxitin Sodium, Cefpodoxime Proxetil, Cefprozil, Ceftaroline Fosamil, Ceftazidime, Ceftibuten, Ceftriaxone Sodium, Cefuroxime Axetil, Cefuroxime Sodium, Celecoxib, Cephalexin, Cephalexin Hydrochloride, Certolizumab Pegol, Cetirizine Hydrochloride, Cetrorelix Acetate, Cevimeline Hydrochloride, Charcoal, Activated, Chenodiol, Chloral Hydrate, Chloramphenicol, Chloramphenicol Sodium Succinate, Chlordiazepoxide, Chlordiazepoxide Hydrochloride, Chlorothiazide, Chlorothiazide Sodium, Chlorpheniramine Maleate, Dexchlorpheniramine Maleate, Chlorpromazine Hydrochloride, Chlorpropamide, Chlorthalidone, Cholestyramine Resin, Choriogonadotropin Alfa, Ciclesonide, Cidofovir, Cimetidine, Cimetidine Hydrochloride, Cinacalcet, Ciprofloxacin, Cisapride, Citalopram Hydrobromide, Clarithromycin, Clemastine Fumarate, Clevidipine Butyrate, Clindamycin Hydrochloride, Clindamycin Palmitate Hydrochloride, Clindamycin Phosphate, Clobazam, Clofazimine, Clomiphene Citrate, Clomipramine Hydrochloride, Clonazepam, Clonidine, Clorazepate Dipotassium, Clozapine, Coccidioidin, Codeine Phosphate, Codeine Sulfate, Colchicine, Colesevelam Hydrochloride, Colestipol Hydrochloride, Colistimethate Sodium, Collagenase Clostridium Histolyticum, Conivaptan Hydrochloride, Corticotropin (Pituitary), Cortisone Acetate, Cosyntropin, Co-trimoxazole, Crofelemer, Cromolyn Sodium, Cycloserine, Cyclosporine, Cyproheptadine Hydrochloride, Dalfampridine, Danazol, Dapsone, Daptomycin, Darifenacin Hydrobromide, Darunavir, Deferasirox, Deferiprone, Deferoxamine Mesylate, Delavirdine Mesylate, Demeclocycline Hydrochloride, Denosumab, Desipramine Hydrochloride, Desloratadine, Desmopressin Acetate, Desvenlafaxine Succinate, Dexamethasone, Dexamethasone Acetate, Dexamethasone Sodium Phosphate, Dexlansoprazole, Dexmedetomidine Hydrochloride, Dexmethylphenidate Hydrochloride, Dexrazoxane Hydrochloride, Dextroamphetamine Saccharate, Dextroamphetamine Sulfate, Dextromethorphan Hydrobromide, Diazepam, Diazoxide, Diclofenac Sodium, Diclofenac Potassium, Diclofenac Epolamine, Dicloxacillin Sodium, Didanosine, Diethylpropion Hydrochloride, Diflunisal, Diltiazem Hydrochloride, Dimenhydrinate, Dimercaprol, Dimethyl Fumarate, Dinoprostone, Diphenhydramine Hydrochloride, Diphenidol Hydrochloride, Diphenoxylate Hydrochloride, Dipyridamole, Disopyramide Phosphate, Disulfuram, Dolasetron Mesylate, Dolutegravir Sodium, Donepezil Hydrochloride, Doripenem, Dornase Alfa, Dosage and Administration, Doxapram Hydrochloride, Doxazosin Mesylate, Doxepin Hydrochloride, Doxycycline Calcium, Doxycycline Hyclate, Doxycycline Monohydrate, Doxylamine Succinate, Dronabinol, Droperidol, Duloxetine Hydrochloride, Dutasteride, Ecallantide, Eculizumab, Edetate Calcium Disodium, Edrophonium Chloride, Efavirenz, Eletriptan Hydrobromide, Elvitegravir and Cobicistat, Emtricitabine, Enalaprilat/Enalapril Maleate, Enfuvirtide, Entecavir, Eplerenone, Epoprostenol Sodium, Eprosartan Mesylate, Ergonovine Maleate, Methylergonovine Maleate, Ertapenem Sodium, Erythromycin, Erythromycin Estolate, Erythromycin Ethylsuccinate, Erythromycin Lactobionate, Erythromycin Stearate, Escitalopram Oxalate, Esmolol Hydrochloride, Esomeprazole Magnesium, Esomeprazole Sodium, Estazolam, Estradiol, Estrogen-Progestin Combinations, Estrogens, Conjugated, USP, Estrogens, Esterified, Estropipate, Eszopiclone, Etanercept, Ethacrynic Acid, Ethacrynate Sodium, Ethambutol Hydrochloride, Ethionamide, Ethosuximide, Ethotoin, Etidronate Disodium, Etodolac, Etomidate, Etravirine, Exenatide, Ezetimibe, Ezogabine, Famciclovir, Famotidine, Febuxostat, Felbamate, Felodipine, Fenofibrate, Fenoldopam, Fenoprofen Calcium, Fentanyl Citrate, Fesoterodine Fumarate, Fexofenadine Hydrochloride, Fidaxomicin, Finasteride, Fingolimod Hydrochloride, Flavoxate Hydrochloride, Flecamide Acetate, Fluconazole, Flucytosine, Fludrocortisone Acetate, Flumazenil, Flunisolide, Fluoxetine Hydrochloride, Fluoxymesterone, Fluphenazine Decanoate, Fluphenazine Hydrochloride, Flurazepam Hydrochloride, Flurbiprofen Sodium, Fluticasone Propionate, Fluvastatin Sodium, Fluvoxamine Maleate, Fomepizole, Fosamprenavir Calcium, Fosinopril Sodium, Fosphenyloin Sodium, Fospropofol Disodium, Frovatriptan Succinate, Furosemide, Gabapentin, Galantamine Hydrobromide, Gallium Nitrate, Galsulfase, Ganciclovir Sodium, Ganirelix Acetate, Gemfibrozil, Gemifloxacin Mesylate, Gentamicin Sulfate, Glatiramer Acetate, Glimepiride, Glipizide, Glucagon, Glucarpidase, Glyburide, Golimumab, Gonadotropin, Chorionic, Granisetron Hydrochloride, Griseofulvin, Guaifenesin, Guanabenz Acetate, Guanfacine, Haloperidol, Haloperidol Decanoate, Haloperidol Lactate, Histoplasmin, Hydralazine Hydrochloride, Hydrochlorothiazide, Hydrocodone Bitartrate, Hydrocortisone, Hydrocortisone Acetate, Hydrocortisone Cypionate, Hydrocortisone Sodium Phosphate, Hydrocortisone Sodium Succinate, Hydromorphone Hydrochloride, Hydroxyprogesterone Caproate, Hydroxyzine Hydrochloride, Hydroxyzine Pamoate, Ibandronate Sodium, Ibuprofen, Icatibant Acetate, Icosapent Ethyl, Idursulfase, Iloperidone, Iloprost, Imiglucerase, Imipenem and Cilastatin, Imipramine Hydrochloride, Imipramine Pamoate, Indapamide, Indigotindisulfonate Sodium, Indinavir Sulfate, Indocyanine Green, Indomethacin, Infliximab, Insulin Aspart, Insulin Detemir, Insulin Glargine, Insulin Glulisine, Insulin Human, Insulin Lispro, Insulin Zinc, Interferon Alfa, Interferon Beta, Interferon Gamma, Irbesartan, Isoniazid, Isosorbide Dinitrate/Mononitrate, Isoxsuprine Hydrochloride, Isradipine, Itraconazole, Ivacaftor, Ivermectin, Kanamycin Sulfate, Ketoconazole, Ketoprofen, Ketorolac Tromethamine, Labetalol Hydrochloride, Lacosamide, Lactobacillus Acidophilus, Lactulose, Lamivudine, Lamotrigine, Lanreotide Acetate, Lansoprazole, Laronidase, Leflunomide, Leucovorin Calcium, Levetiracetam, Levocetirizine Dihydrochloride, Levofloxacin, Levoleucovorin Calcium, Levomilnacipran Hydrochloride, Levorphanol Tartrate, Levothyroxine Sodium, Lidocaine Hydrochloride, Linaclotide, Linagliptin, Lincomycin Hydrochloride, Linezolid, Liothyronine Sodium, Liotrix, Liraglutide, Lisdexamfetamine Dimesylate, Lisinopril, Lithium Salts, Loperamide Hydrochloride, Lopinavir and Ritonavir, Loratadine, Lorazepam, Lorcaserin Hydrochloride, Losartan Potassium, Lovastatin, Loxapine Succinate, Lubiprostone, Lurasidone Hydrochloride, Lutropin Alfa, Macitentan, Magnesium Sulfate, Mannitol, Maprotiline Hydrochloride, Maraviroc, Mebendazole, Mecasermin, Meclizine Hydrochloride, Meclofenamate Sodium, Medroxyprogesterone Acetate, Mefenamic Acid, Meloxicam, Memantine Hydrochloride, Meperidine Hydrochloride, Mephobarbital, Meprobamate, Meropenem, Mesalamine, Mesna, Metformin Hydrochloride, Methadone Hydrochloride, Methamphetamine Hydrochloride, Methimazole, Methohexital Sodium, Methsuximide, Methyclothiazide, Methyldopa, Methyldopate Hydrochloride, Methylene Blue, Methylnaltrexone Bromide, Methylphenidate Hydrochloride, Methylprednisolone, Methylprednisolone Acetate, Methylprednisolone Sodium Succinate, Methyltestosterone, Metoclopramide Hydrochloride, Metolazone, Metoprololol Succinate, Metoprolol Tartrate, Metyrapone, Metyrosine, Mexiletine Hydrochloride, Micafungin Sodium, Midazolam Hydrochloride, Mifepristone, Miglitol, Miglustat, Milnacipran Hydrochloride, Mineral Oil, Minocycline Hydrochloride, Minoxidil, Mirabegron, Mirtazapine, Misoprostol, Modafinil, Moexipril Hydrochloride, Molindone Hydrochloride, Mometasone Furoate, Montelukast Sodium, Morphine Sulfate, Morrhuate Sodium, Moxifloxacin Hydrochloride, Mumps Skin Test Antigen, Mycophenolate, Nabilone, Nabumetone, Nadolol, Nafarelin Acetate, Nafcillin Sodium, Nalbuphine Hydrochloride, Nalmefene Hydrochloride, Naloxone Hydrochloride, Naltrexone, Naproxen, Naproxen Sodium, Naratriptan Hydrochloride, Natalizumab, Nateglinide, Nebivolol Hydrochloride, Nefazodone Hydrochloride, Nelfinavir Mesylate, Neomycin Sulfate, Neostigmine Bromide, Neostigmine Methylsulfate, Nesiritide, Nevirapine, Niacin, Nicardipine, Nifedipine, Nimodipine, Nisoldipine, Nitisinone, Nitric Oxide, Nitroglycerin, Nizatidine, Norethindrone Acetate, Norfloxacin, Nortriptyline Hydrochloride, Nystatin, Octreotide Acetate, Ofloxacin, Olanzapine, Olanzapine Pamoate, Olmesartan Medoxomil, Olsalazine Sodium, Omalizumab, Omega-3-acid Ethyl Esters, Omeprazole, Omeprazole Magnesium, Ondansetron Hydrochloride, Opium, Orlistat, Oseltamivir Phosphate, Ospemifene, Oxacillin Sodium, Oxandrolone, Oxaprozin, Oxaprozin Potassium, Oxazepam, Oxcarbazepine, Oxybutynin Chloride, Oxycodone, Oxycodone Hydrochloride, Oxycodone Terephthalate, Oxymorphone Hydrochloride, Oxytocin, Paliperidone, Palivizumab, Palonosetron Hydrochloride, Pamidronate Disodium, Pancrelipase, Pantoprazole Sodium, Papaverine Hydrochloride, Paroxetine, Pasireotide Diaspartate, Peginterferon Alfa, Pegloticase, Pegvisomant, Penicillamine, Penicillin G Benzathine, Penicillin G Potassium, Penicillin G Sodium, Penicillin G Procaine, Penicillin V, Penicillin V Potassium, Pentazocine Hydrochloride, Pentazocine Lactate, Pentobarbital, Pentobarbital Sodium, Perindopril Erbumine, Perphenazine, Pharmacology, Phendimetrazine Tartrate, Phenelzine Sulfate, Phenobarbital, Phenobarbital Sodium, Phentermine, Phentermine Hydrochloride, Phenyloin, Phenyloin Sodium, Physostigmine Salicylate, Pimozide, Pindolol, Pioglitazone Hydrochloride, Piperacillin Sodium and Tazobactam Sodium, Piroxicam, Pitavastatin Calcium, Polidocanol, Polymyxin B Sulfate, Polythiazide, Poractant Alfa, Posaconazole, Potassium Iodide, Pralidoxime Chloride, Pramlintide Acetate, Pravastatin Sodium, Praziquantel, Prazosin Hydrochloride, Prednisolone, Prednisolone Sodium Phosphate, Prednisone, Pregabalin, Preparations, Primidone, Probenecid, Procainamide Hydrochloride, Prochlorperazine, Prochlorperazine Edisylate, Prochlorperazine Maleate, Progesterone, Progestins (Etonogestrel, Levonorgestrel, Norethindrone), Promethazine Hydrochloride, Propafenone Hydrochloride, Propofol, Propranolol Hydrochloride, Propylthiouracil, Protriptyline Hydrochloride, Pyrantel Pamoate, Pyrazinamide, Pyridostigmine Bromide, Quazepam, Quetiapine Fumarate, Quinapril Hydrochloride, Quinidine Gluconate, Quinidine Sulfate, Quinupristin and Dalfopristin, Rabeprazole Sodium, Raloxifene Hydrochloride, Raltegravir Potassium, Ramelteon, Ramipril, Ranitidine Hydrochloride, Rasburicase, Rauwolfia Alkaloids, Regadenoson, Remifentanil Hydrochloride, Repaglinide, Ribavirin, Rifabutin, Rifampin, Rifapentine, Rifaximin, Rilonacept, Rilpivirine Hydrochloride, Riluzole, Rimantadine Hydrochloride, Riociguat, Risedronate Sodium, Risperidone, Ritonavir, Rivastigmine, Rivastigmine Tartrate, Rizatriptan Benzoate, Roflumilast, Rosiglitazone Maleate, Rosuvastatin Calcium, Rufinamide, Sacrosidase, Salicylamide, Salicylate Salts, Salsalate, Sapropterin Dihydrochloride, Saquinavir Mesylate, Saxagliptin Hydrochloride, Secobarbital, Secobarbital Sodium, Secretin, Sertraline Hydrochloride, Sildenafil Citrate, Simethicone, Simvastatin, Sincalide, Sirolimus, Sitagliptin Phosphate, Sodium Bicarbonate, Sodium Chloride 20% Injection, Sodium Lactate, Sodium Nitroprusside, Sodium Oxybate, Sodium Phenylacetate and Sodium Benzoate, Sodium Phenylbutyrate, Sodium Tetradecyl Sulfate, Sodium Thiosalicylate, Solifenacin Succinate, Sotalol Hydrochloride, Spectinomycin Hydrochloride, Spironolactone, Stavudine, Stool Softeners, Streptomycin Sulfate, Succimer, Sucralfate, Sufentanil Citrate, Sulfadiazine, Sulfasalazine, Sulfinpyrazone, Sulindac, Sumatriptan, Sumatriptan Succinate, Tacrine Hydrochloride, Tacrolimus, Tadalafil, Talc, Taliglucerase Alfa, Tapentadol Hydrochloride, Teduglutide, Tegaserod Maleate, Telavancin Hydrochloride, Telbivudine, Telithromycin, Telmisartan, Temazepam, Tenofovir Disoproxil Fumarate, Terazosin Hydrochloride, Terbinafine Hydrochloride, Teriflunomide, Teriparatide, Tesamorelin Acetate, Testosterone, Testosterone Cypionate, Testosterone Enanthate, Testosterone Propionate, Tetrabenazine, Tetracycline, Tetracycline Hydrochloride, Thalidomide, Theophyllines, Thiopental Sodium, Thioridazine Hydrochloride, Thiothixene, Thyroid, Tiagabine Hydrochloride, Ticarcillin Disodium and Clavulanate Potassium, Tigecycline, Timolol Maleate, Tipranavir, Tobramycin Sulfate, Tocilizumab, Tofacitinib Citrate, Tolazamide, Tolbutamide, Tolmetin Sodium, Tolterodine Tartrate, Tolvaptan, Topiramate, Torsemide, Tramadol Hydrochloride, Trandolapril, Tranylcypromine Sulfate, Trazodone Hydrochloride, Treprostinil, Triamcinolone Triamcinolone Acetonide, Triamcinolone Diacetate, Triamcinolone Hexacetonide, Triamterene, Triazolam, Trifluoperazine Hydrochloride, Trimethobenzamide Hydrochloride, Trimipramine Maleate, Triprolidine Hydrochloride, Tromethamine, Trospium Chloride, Tuberculin, Ulipristal Acetate, Urea, Urea 40-50% Injection, Ursodiol, Valacyclovir Hydrochloride, Valganciclovir Hydrochloride, Valproate Sodium, Valproic Acid, Divalproex Sodium, Valsartan, Vancomycin Hydrochloride, Vardenafil Hydrochloride, Vasopressin, Velaglucerase Alfa, Venlafaxine Hydrochloride, Verapamil Hydrochloride, Vigabatrin, Vilazodone Hydrochloride, Voriconazole, Xylose, Zafirlukast, Zaleplon, Zanamivir, Ziconotide, Zidovudine, Zileuton, Ziprasidone, Zoledronic Acid, Zolmitriptan, Zolpidem Tartrate, Zonisamide, and α1-Proteinase Inhibitor (Human).

The phrase “pharmaceutical combinations” is meant for purposes of the present invention to mean a pharmaceutical drug or medicinal product or any chemical substance formulated or compounded as a combination of pharmacologically active substances, as a combination product intended for internal, or external or for use in the medical diagnosis, cure, treatment, or prevention of disease.

The phrase “metabolite formation” is meant for purposes of the present invention to mean the method of quantifying the rate of a metabolite appearance or formation, as a result of a chemical reaction, in a given system over a specified period of time.

The phrase “comparing metabolite formation” is meant for purposes of the present invention to mean a test to determine relatedness or similarity between experimental or treatment group and control groups with respect to formation of a given metabolite.

The term “cell” is meant for purposes of the present invention to mean the basic structural, functional, and biological unit of all known living organisms, and the smallest unit of life that can replicate independently. The term kidney cells are is meant for purposes of this invention to mean cells derived from a mammalian kidney or kidney structure. For purposes of this invention, human kidney cells are meant to mean cells derived from the a human kidney or kidney structure.

The phrase “control group” is meant for purposes of the present invention to mean that group in a comparative experiment that receive either no treatment or a standard treatment

The phrase “treatment group” is meant for purposes of the present invention to mean that group in a comparative experiment that receive a pre-determined exposure or standard treatment.

The phrase “exposing cells” is meant for purposes of the present invention to mean the process of direct contact between a cell with an active agent or pharmaceutical over a specified period of time on either an in vitro or in vivo basis.

The term “comparing” is meant for purposes of the present invention to mean a test to determine relatedness or similarity between experimental treatment group and control groups.

The phrase “making a determination” is meant for purposes of the present invention to mean the process of quantifying a given sample or group for the purpose of comparison to a control or other group.

The term “HPLC” is meant for purposes of the present invention to mean high performance liquid chromatography and related techniques known to those of ordinary skill in the art in analytic chemistry used to separate components in a mixture, to identify each component, and to quantify each component.

The phrase “period of time” is meant for purposes of the present invention to mean the pre-specified time to conduct a given experiment. One such period of time comprises about 0-18 hours and any time period or interval between this time.

The term “inhibitors” is meant for purposes of the present invention to mean pharmaceutical products or substances that reduce or eliminate the activity of a given biological system, such as enzymes or transporters.

The term “inducers” is meant for purposes of the present invention to mean pharmaceutical products or substances that elevate the activity of a given biological system, such as enzymes or transporters

The phrase “NAT enzyme” is meant for purposes of the present invention to mean N-acetyltransferase (NAT) that catalyzes the transfer of acetyl groups from acetyl-CoA to arylamines.

The term “unknown” is meant for purposes of the present invention to mean a variable substance appearing in an equation or body system, in which the equation has to be solved.

The phrase “nephrotubular metabolic activity” is meant for purposes of the present invention to mean measurement of cumulative urinary excretion of substances filtered, secreted or absorbed in the nephron, through use of endogenous biomarkers or exogenous diagnostic agents.

The term “samples” is meant for purposes of the present invention to mean a limited quantity of a biological or experimental matrix which is intended to be similar to and represent a larger amount of that matrix.

The phrase “quantifying acetyl-PAH” is meant for purposes of the present invention to mean the method of calculating the amount of acetyl-PAH appearing in a given biological or experimental matrix over a specified period of time, often employing chemical analysis techniques such as HPLC.

The phrase “diagnostic agent” is meant for purposes of the present invention to mean an endogenous or exogenous chemical substance used to reveal, identify, and define the localization or function of a pathological or physiological process.

The phrase “first reading” is meant for purposes of the present invention to mean the method of quantifying a biological activity at baseline or the starting point used for comparison in a given experiment.

The phrase “second reading” is meant for purposes of the present invention to mean the method of quantifying a biological activity after a specified period of time after the first reading, for comparison in a given experiment.

The phrase “iothalamate infusion or iohexyl” is meant for purposes of the present invention to mean the method of determining glomerular filtration rate (GFR) in a biological system using the diagnostic agents iothalamate or iohexyl, often given as in intravenous injection or infusion.

The phrase “plasma samples” is meant for purposes of the present invention to mean a limited quantity of human plasma which is intended to be similar to and represent a larger amount of that matrix.

The phrase “urine samples” is meant for purposes of the present invention to mean a limited quantity of human urine which is intended to be similar to and represent a larger amount of that matrix.

The phrase “time points” is meant for purposes of the present invention to mean the pre-specified times that plasma or urine samples are drawn from a subject during the conduct a given experiment.

The term “administration” is meant for purposes of the present invention to mean the process of delivering a pharmaceutical or diagnostic agent on an in-vitro and/or in-vivo basis. The phrase “statistical comparison” is meant for purposes of the present invention to mean the process of using a set of statistical inferences about a subset of parameters selected based on the observed values and comparison of groups to determine the degree of difference, if present.

One embodiment of the present invention comprises a method of determining nephrotoxicity of pharmaceuticals, comprising the steps of conducting a metabolite formation study using PAH in Control Group A (negative control); measuring metabolite formation in Control Group A using HPLC; exposing kidney cells to nephrotoxic intervention or insult in Treatment Group B over a specified period of time; conducting metabolite formation study using PAH in Treatment Group B using HPLC, and statistically comparing metabolite formation in Group A vs. Group B

Another embodiment of the present invention comprises a method of screening for drug interactions (multiple combinations), comprising the steps of conducting metabolite formation study using PAH and Drug X in Control Group A; measuring metabolite formation in Control Group A; exposing kidney cells to interacting drug or combination of drugs Treatment Group B over a specified period of time to inhibit or induce PAH uptake into kidney cells; conducting metabolite formation study using PAH in Treatment Group B; and, statistically comparing metabolite formation in Group A vs. Group B.

Another embodiment of the present invention comprises a method of determining nephrotubular metabolic activity, comprising the steps of enrolling human subjects based on a set of inclusion/exclusion criteria; designing PAH and iothalamate infusion or iohexyl injection regimen based in estimated GFR of subject; using iothalamate or iohexyl for glomerular filtration rate (GFR) measurement during PAH infusion; sampling blood for infusion of PAH/IOTH solution; administering PAH and IOTH or as a sequential dose-escalation infusion; collecting plasma and urine samples at specified time points during the infusions; quantifying acetyl-PAH and iothalamate or iohexyl using HPLC methods; administering one or more pharmaceuticals that inhibit or induce metabolic activity over a specified period of time; repeating these steps; and, statistically comparing acetyl-PAH formation in a pre vs. post (paired) within-subject analysis.

Another embodiment of the present invention comprises a method wherein PAH and/or iothalamate and/or iohexyl is administered at various times in a longitudinal fashion (such as every 6 months or yearly) to evaluate tubular metabolic activity and GFR over time.

Another embodiment of the present invention comprises a method of determining nephrotoxicity of pharmaceuticals, the method comprises conducting a metabolite formation study in cells using PAH in a control group, measuring metabolite formation in the control group, exposing cells to pharmaceuticals in a treatment group, conducting metabolite formation study using PAH in the treatment group, measuring metabolite formation in the treatment group, comparing metabolite formation in the control group and the treatment group, and making a determination as to the nephrotoxicity of the pharmaceutical. In this method, metabolite formation may comprise measuring using HPLC. In this method, the step of exposing cells to pharmaceuticals may occur over a period of time comprising about 0-18 hours. In this method, the cells may comprise kidney cells that are animal or human kidney cells. In this method, the comparison may comprise a statistical comparison.

Another embodiment of the present invention comprises a method of determining nephrotoxicity of pharmaceutical combinations, the method comprising conducting a metabolite formation study in cells using PAH and a first pharmaceutical in control group, measuring metabolite formation in the pharmaceutical control group, exposing said cells to one or more pharmaceuticals in a treatment group, measuring metabolite formation in the treatment group, comparing metabolite formation in the control group and the treatment group, and determining the nephrotoxicity of the pharmaceutical combinations. In this method, the metabolite formation study comprises using PAH and a first pharmaceutical in control group and comprises conduct it in kidney cells. In this method, the step of exposing cells to pharmaceuticals comprises exposure over a period of time. In this method, the pharmaceuticals may comprise inhibitors or inducers of a NAT enzyme. In this method it is unknown whether the pharmaceuticals may comprise inhibitors or inducers of PAH uptake into kidney cells of a NAT enzyme. In this method, the comparison may comprise a statistical comparison.

Another embodiment of the present invention comprises a method of determining nephrotubular metabolic activity, the method comprises administering PAH and a diagnostic agent to a human, collecting samples from the human, quantifying acetyl-PAH and the diagnostic agent and establishing a first reading, administering a pharmaceutical to the human, collecting samples from the human, comparing acetyl-PAH formation, quantifying acetyl-PAH and the pharmaceutical and establishing a second reading, comparing acetyl-PAH formation between the first and second readings, determining the change in nephrotubular metabolic activity between the first and second readings. This method may comprise a diagnostic agent comprising iothalamate infusion or iohexyl. This method may comprise samples comprising plasma or urine samples. This method may comprise the collection of samples at times points during the infusions. This method may comprise collecting plasma or urine samples during and after said administration of said pharmaceutical. This method may comprise administering PAH and a diagnostic agent to a human based on the glomerular infusion rate of the human. This method may comprise a comparison comprising a statistical comparison.

### Example 1

### Synthesis of Acetyl-PAH for Assay Quantification

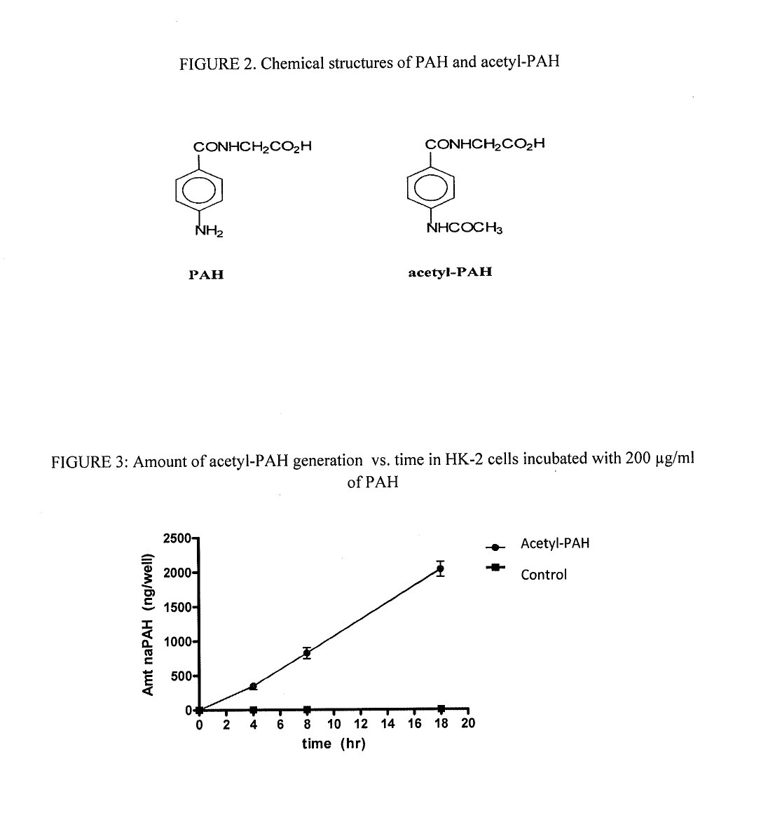

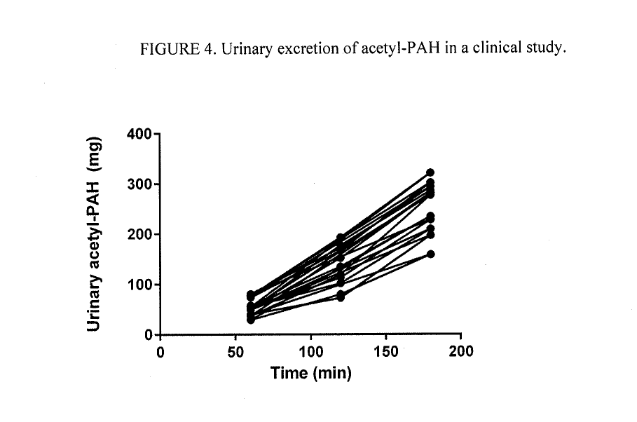

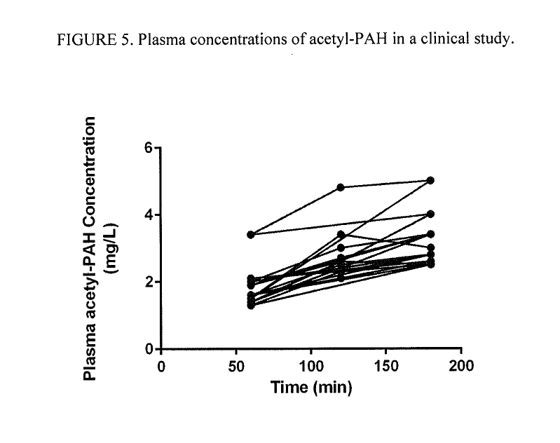

Acetyl-PAH (FIG. 2) was synthesized by combining 1.9 mL of acetic anhydride with 20 mL of a 1% PAH solution, and allowed to stand at room temperature for 30 minutes with occasional shaking. The mixture was cooled in an ice bath, filtered by suction through a Buchner funnel and washed several times with ice-cold deionized, distilled water followed by 95% ethanol. The precipitate was dried on filter paper. The purity was confirmed by LC-MS.

### Example 2

### HPLC Quantification of Acetyl-PAH

Acetyl-PAH was quantified by HPLC using a Waters 2690 Separation Module. Analyte separation was achieved with a C18 125A 10μ 300 mm column (Grace Discovery, Deerfield, Ill.) with an acetonitrile-based mobile phase and UV detection at 254 nm. The runtime was 25 minutes for all samples. The HPLC results showed that mean (±SD) naPAH metabolite concentration increased over time (FIG. 2) The negative control showed absence of acetyl-PAH (FIG. 3).

### Example 3

### Cell Culture

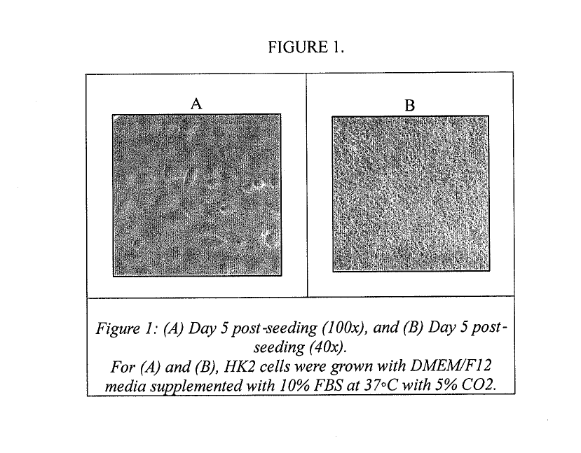

HK2 cells were grown in 15 ml of Dulbecco's Modified Eagle Medium (DMEM)/F12 media with 10% Fetal Bovine Serum (FBS) and incubated at 37° C. with 5% CO₂. After 5 days of culture, each 75 cm² flask of cells was transferred to 2 mls of the culture media and incubated with 200 μg/ml of PAH except for the negative control where no PAH was added. Cells were incubated for 4, 8 and 18 hours each (in triplicate) with or without PAH (control). Cells post incubation were lysed via sonication for 1 minute, then centrifuged at 14,000 RPM for 10 minutes. The supernatant was extracted and acetyl-PAH was quantified by HPLC. After 5 days of culture, the HK2 cell monolayers reached confluence (FIG. 1).

### Steps for Thawing Cells:

1. Warm DMEM/F12 media and DFBS in 37° C. water bath for 30 minutes
2. Turn on UV lamp in cell culture hood for 30 minutes
3. Spray down hood and media, DFBS and trypsin bottles with 70% ethanol and wipe clean
4. Add warmed 50 ml of FBS, 5 ml of P/S and 8.25 ml of HEPES buffer to the warmed 500 ml bottle of DMEM/F12 to make the media
5. Remove cells from the liquid nitrogen, and thaw by holding the cryogenic tube in 37° C. water bath for 1 minute.
6. Transfer entire content of cryogenic tube to 10 ml of pre-warmed media in a 15 ml test tube
7. Gently invert five times, then spin at 8000 RPM for 10 minutes.
8. Discard supernatant making sure not to disturb the pellet, then add 15 ml of media to the test tube
9. Invert five times, making sure the pellet is dissolved within the media, then plate the entire contents onto a 75 cm³ flask by pipetting all 15 mls onto the cell culture plate
10. Incubate at 37° C. with 5% CO₂ for 5 days

### Growing Cells (5-Day Cycle)

1. Warm media and DPBS in 37° C. water bath for 30 minutes, warm the trypsin digest to room temperature for 30 minutes
2. Turn on UV lamp in cell culture hood for 30 minutes
3. Spray down hood and media, DFBS and trypsin bottles with 70% ethanol and wipe clean
4. Remove cells from incubator and discard old media, be careful not to disturb the layer of cells growing on the bottom of the flask
5. Add 10 ml of warmed DPBS buffer to the flask and gently swirl.
6. Remove and discard DPBS buffer, add 2 ml trypsin digest buffer to flask, wait 2 minutes
7. Remove and discard trypsin digest buffer making sure not to disturb the bottom of the flask
8. Add 10 ml of media to flask and pipette the media 5 times over bottom of cell culture flask (using electronic pipette) to detach cells
9. Place 2 ml of cell culture media and 13 ml of fresh media in a new flask for a 1:5 dilution of cells.
10. Repeat step 9 in new clean flask

Incubate both flasks from steps 9 and 10 at 37° C. with 5% CO₂ for 5 days

### HPLC

1. The mobile phase consists of 120 ml MeOH, 30 ml acetonitrile, 20 ml 1M Sodium Acetate pH=5, 209.6 mg TBA, QS 1 L of deionized H₂O
2. May use a C18 125A 10μ 300 mm column
3. Set runtime at 14 minutes for all samples.
4. Add 100 μL of thawed supernatant from step 7 in PAH metabolism section to HPLC tubes.

### Example 4

### PAH Metabolism Experiment

1. HK2 cells were grown in 15 ml of Dulbecco's Modified Eagle Medium (DMEM)/F12 media with 10% Fetal Bovine Serum (FBS) and incubated at 37° C. with 5% CO₂. After 5 days of culture, each 75 cm² flask of cells was transferred to 2 mls of the culture media and incubated with 200 μg/ml of PAH except for the negative control (no PAH was added). Cells were incubated for 4, 8 and 18 hours each (in triplicate) with or without PAH (control). Add 2 mls of media with 200 μg/ml of PAH (40 μl of stock PAH) to the flask, pipette the media 5 times over the flask
2. Transfer cells and media from the flask to a 15 ml test tube
3. Incubate cells at 37° C. for set period of time with a control tube of no PAH
4. Lyse cells by sonification by holding test tube in sonicator for 1 minute
5. Centrifuge ×14,000 RPM for 10 minutes
6. Extract supernatant and store at −20° C. until analysis.

Acetyl-PAH was quantified by HPLC using a Waters 2690 Separation Module. Analyte separation was achieved with a C18 125A 10μ 300 mm column with an acetonitrile-based mobile phase and UV detection at 254 nm. The retention time for acetyl-PAH was approximately 11 minutes, and the runtime was 14 minutes for all samples. The acetyl-PAH limit of detection was 20 ng/mL. Quantification of acetyl-PAH formation was determined by calculating the area under the curve for each sample time-point, and determining the cumulative appearance over the entire incubation period (up to 18 hours).

### Example 5

### Western Blot Experiment

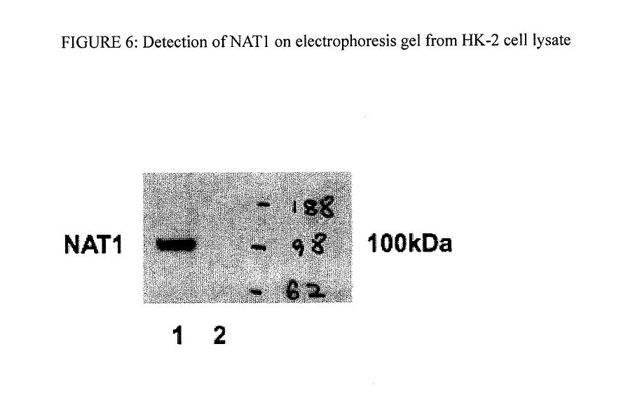

HK-2 cells were grown in 15 ml of Dulbecco's Modified Eagle Medium (DMEM)/F12 media with 10% Fetal Bovine Serum (FBS) and incubated at 37° C. with 5% CO₂. After 5 days of incubation, cells were harvested and placed in lysis buffer (Cell Lysis Buffer from Cell Signaling Technology, catalog #9803). Cells were lysed via sonification, and resulting protein samples were run on an electrophoresis gel, transferred to a membrane and blocked with 5% milk. The membrane was then treated with 0.5 mcg/mL of primary NAT1 antibody (Sigma-Aldrich) in 1% milk overnight. The next day, the membrane was washed and treated with anti-mouse secondary antibody in 1% milk for 1 hour, treated with reagent and exposed to photographic film for 24 hours. Expression of NAT1 is shown in FIG. 6.

Results from Cell Culture Studies:

This model shows that HK2 cells have functional metabolism via induction or activation of N-acetyltransferase in the presence of PAH in HK-2 cells.

HK2 cells metabolize PAH in a time dependent fashion, as more drug was metabolized to acetyl-PAH the longer the cells were allowed to incubate with PAH.

### Example 6

### Measurement of Urinary N-Acetyl-Transferase Activity Human Protocol

The purpose of this study is to quantify plasma and urinary acetyl-PAH as a measure of tubular function, in the presence of simultaneous measurement of GFR (iothalamate clearance) in healthy subjects. Following the informed consent process, each subject was evaluated for past medical history, had a physical examination and routine laboratory tests (including urinary protein:creatinine ratio) within 4 weeks prior to the study visit. Subjects receiving drugs known to interfere with tubular secretion based on a current review of available literature, or a history of known allergic or adverse reactions to diagnostic iodine containing compounds including iothalamate, or PAH were excluded. Subjects with preexisting liver disease (including hepatitis), increased INR, liver transaminases (AST/ALT), bilirubin, serum albumin<3.0 g/dL. Iothalamate (Conray®, Mallinkrodt), and PAH (Merck) infusions were prepared on the morning of the study visit.

Study Visit:

On the study day, each subject reported to the General Clinical Research Unit (GCRC) by 8:00 AM on study visit days. A 6-mL blood sample was be obtained for biochemistries (BUN, serum creatinine) and a spot urine sample was obtained for protein:creatinine ratio. Vital signs (heart rate, blood pressure, respiratory rate) were recorded half-hourly throughout each study visit. Subjects had intravenous catheters inserted into forearm veins of each arm for blood collection and intravenous infusion of iothalamate and PAH. From 30 minutes before marker administration until the end of each evaluation period, subjects remained in a semi-reclined position except during urine collections to minimize the effects of changes in posture on GFR. Subjects were given a water loading regimen, consisting of 250 mL of water at 30 minutes and 15 minutes, respectively, prior to the start of the infusions. Combined oral or IV fluids (5% dextrose) were then given to maintain fluid intake equal to urine output from each preceding half-hour during the next 10 minutes of the subsequent urine collection throughout the remainder of the study day. Initial priming doses were administered over 5 minutes consisting of 2 mL (456 mg), and 1 mL (200 mg) for iothalamate and PAH, respectively. A constant-rate infusion was then initiated at 1 mL/min over 180 minutes, with the concentration in the infusate determined based on the patient's estimated renal clearance and target plasma concentration of 10 mg/L and 15 mg/L for iothalamate, and PAH, respectively. The study procedures are shown in the table below:

### Clinical Study Procedures

| | | Blood Draws | Urine Collection |
|---|---|---|---|
| | Drug Administration | B = 7 mL | U = 5 mL urine |
| Time | IOTH = iothalamate | green top tube | aliquot |
| Baseline | — | B (predose) | U (predose) |
| (−0.5 hr) | | | |
| 0 to +3 hr | Give IOTH, PAH bolus | B (every 30 | U (every 30 |
| | Start IOTH, PAH | min) | min) |
| | infusions (over 4 hours) | | |

Sample Collection and Processing:

Throughout the study, all blood samples were collected in heparinized (green top) tubes, placed on ice immediately, centrifuged within 15 minutes (4,000 RPM), split into 2 plasma aliquots and frozen at −20° C. until analyzed. Urine was collected at baseline and in 30 minute intervals throughout each study visit. Urine was be placed on ice immediately after collection, and three 8 mL aliquots were frozen at −20° C. until analyzed. The total volume of urine was measured for each collection interval. A 5 mL aliquot of each IV infusate was collected and frozen at −20° C. until analyzed.

### Example 7

### Analytical Techniques

Iothalamate and PAH: Iothalamate and PAH concentrations in plasma and urine will be determined using an HPLC assay method developed in our laboratory. Briefly, the method involves deproteinizing plasma samples with acetonitrile followed by evaporation and reconstitution in mobile phase. Urine samples are diluted in mobile phase prior to injection. Separation is achieved using a C₁₈ column, and effluent is monitored at a wavelength of 254 ηm. The within and between day coefficients of variation for this assay are less than 10%.

### Example 8

### Pharmacokinetic Data Analysis

The primary outcome variables were plasma acetyl-PAH and PAH concentrations, cumulative urinary excretion of acetyl-PAH and PAH, renal clearance of acetyl-PAH(CLr_aPAH) and PAH (CLr_PAH), and renal clearance of iothalamate (GFR). Each of these indices was calculated using standard pharmacokinetic equations. The rate of tubular secretion for each is then estimated as follows: R_{sec}=R_{exc}−(GFR*Cu) where GFR is renal clearance of iothalamate, Cu is the plasma concentration (acetyl-PAH) and R_{sec} is the total rate of tubular secretion of aPAH and PAH, and R_{exc} is the net urinary excretion rate of aPAH and PAH. The results showed that the GFR of the healthy volunteers was 111±56 mL/min. The renal clearance (CLr) of PAH was 493±196 and the CLr of acetyl-PAH was 692±207 mL/min. The Rsec was higher for acetyl-PAH compared to PAH (581±191 mL/min vs. 382±154 mL/min, respectively).

Results from Human Studies:

When PAH is administered to humans, the acetyl-PAH metabolite appears in high quantities in the urine and plasma indicating activity of N-acetyl transferase enzyme.

1. A method of determining nephrotoxicity of pharmaceuticals, said method comprises conducting a metabolite formation study in cells using PAH in a control group, measuring metabolite formation in said control group, exposing cells to pharmaceuticals in a treatment group, conducting metabolite formation study using PAH in said treatment group, measuring metabolite formation in said treatment group, comparing metabolite formation in said control group and said treatment group, and making a determination as to the nephrotoxicity of said pharmaceutical.

2. The method according to claim 1, wherein metabolite formation comprises measuring using HPLC.

3. The method according to claim 1, wherein the step of exposing cells to pharmaceuticals occurs over a period of time.

4. The method according to claim 3, wherein said period of time comprises about 0-18 hours.

5. The method according to claim 1 wherein said cells comprise kidney cells.

6. The method of claim 5, wherein said kidney cells comprise human kidney cells.

7. The method of claim 1, wherein said comparison comprises a statistical comparison.

8. A method of determining drug interactions of pharmaceutical combinations, said method comprising conducting a metabolite formation study in cells using PAH and a first pharmaceutical in control group, measuring metabolite formation in said pharmaceutical control group, exposing said cells to one or more pharmaceuticals in a treatment group, measuring metabolite formation in said treatment group, comparing metabolite formation in said control group and said treatment group, and determining the drug interactions of said pharmaceutical combinations.

9. The method according to claim 8, wherein said metabolite formation study comprises using PAH and a first pharmaceutical in control group, and is conducted in kidney cells.

10. The method according to claim 8, wherein the step of exposing cells to pharmaceuticals occurs over a period of time.

11. The method according to claim 8, wherein said pharmaceuticals comprise inhibitors or inducers of a NAT enzyme.

12. The method according to claim 8, wherein it is unknown whether said pharmaceuticals comprises inhibitors or inducers of PAH uptake into kidney cells of a NAT enzyme.

13. The method according to claim 8, wherein said comparison comprises a statistical comparison.

14. A method of determining nephrotubular metabolic activity, said method comprising administering PAH and a diagnostic agent to a human, collecting samples from said human, quantifying acetyl-PAH and said diagnostic agent and establishing a first reading, administering a pharmaceutical to said human, collecting samples from said human, comparing acetyl-PAH formation, quantifying acetyl-PAH and said pharmaceutical and establishing a second reading, comparing acetyl-PAH formation between said first and second readings, determining the change in nephrotubular metabolic activity between said first and second readings.

15. The method according to claim 14, wherein said diagnostic agent comprises iothalamate infusion or iohexyl.

16. The method according to claim 14, wherein said samples comprises plasma or urine samples.

17. The method according to claim 14, wherein said samples are collected at times points during said infusions.

18. The method according to claim 16, wherein said plasma or urine samples are collected during and after said administration of said pharmaceutical.

19. The method according to claim 14, wherein said PAH and a diagnostic agent(s) are administered to a human based on the glomerular infusion rate of said human.

20. The method according to claim 14, wherein said comparison comprises a statistical comparison.
